(19) United States
(12) Patent Application Publication
(10) Pub. No.: US 2026/0199040 A1
(43) Pub. Date: Jul. 16, 2026

(54) ROBOTIC SURGICAL SYSTEM, SURGICAL ROBOT, METHOD FOR CONTROLLING ROBOTIC SURGICAL SYSTEM, AND STORAGE MEDIUM

(71) Applicant: KAWASAKI JUKOGYO KABUSHIKI KAISHA, Kobe-shi (JP)

(72) Inventors: Ayataka KOBAYASHI, Kobe-shi (JP); Kazuki KODAMA, Kobe-shi (JP)

(73) Assignee: KAWASAKI JUKOGYO KABUSHIKI KAISHA, Kobe-shi (JP)

(21) Appl. No.: 19/383,281

(22) Filed: Nov. 7, 2025

(30) Foreign Application Priority Data
Nov. 14, 2024 (JP) 2024-198807

(51) Int. Cl. A61B 34/37; A61B 34/20

(52) U.S. Cl. CPC A61B 34/37; A61B 34/20

(57) ABSTRACT

In a robotic surgical system according to this disclosure, a controller performs return processing, if a surgical instrument is deviated from a pivot position stored in a storage, to return a robot arm to a position that makes the surgical instrument pass through the pivot position at a moving speed smaller than a moving speed of the robot arm moved through operation of an operation tool.

### CROSS-REFERENCE TO RELATED APPLICATION

The priority application number JP2024-198807, robotic surgical system, surgical robot, method for controlling robotic surgical system, and storage medium, Nov. 14, 2024, Ayataka Kobayashi and Kazuki Kodama, upon which this patent application is based, are hereby incorporated by reference.

### BACKGROUND OF THE INVENTION

### Field of the Invention

The present disclosure relates to a robotic surgical system, a surgical robot, a method for controlling a robotic surgical system, and a storage medium.

### Description of the Background Art

Robotic surgical systems including robot arms to which surgical instruments are attached are known in the art. Japanese Patent Publication No. JP 7176037 discloses a robotic surgical system including a robot provided with a robot arm to which a surgical instrument is attached and which includes joints. Here, external disturbances that act on the robot arm may cause deviation of the surgical instrument inserted into a patient. The deviation of surgical instrument adversely affects the patient. To address this, joints of the robot arm are driven to hold the surgical instrument or the like at a predetermined predetermined position, which is previously set, even when the robot arm is subjected to external disturbances in Japanese Patent Publication No. JP 7176037. Accordingly, the deviation of the surgical instrument inserted into the patient can be reduced.

In the robotic surgical system of Japanese Patent Publication No. JP 7176037, an operation tool is provided to allow an operator to move the robot arm. Here, during the operator moves the robot arm through operation of the operation tool, when the joints of the robot arm are driven to hold the robot arm or the like at the predetermined position, which is previously set, as in Japanese Patent Publication No. JP 7176037, the joints of the robot arm may be driven in a direction different from a direction in which the operator intends to move the robot arm. In this case, because the robot arm moves in a direction different from the operator's intended direction, such movement may confuse the operator.

### SUMMARY OF THE INVENTION

The present disclosure provides a robotic surgical system, a surgical robot, a method for controlling a robotic surgical system, and a storage medium capable of preventing confusion of an operator caused by movement of a robot arm in a direction different from the operator's intended direction when the operator moves the robot arm through operation of an operation tool.

A robotic surgical system according to a first aspect of the present disclosure includes a robot arm having a distal end to which a surgical instrument is attached; a pivot-position setter that stores, in a storage, a pivot position on the surgical instrument attached to the robot arm that serves as a pivot point during movement; an operation tool that controls movement of the surgical instrument moved by the robot arm; and a controller that performs return processing, if the surgical instrument is deviated from the pivot position stored in the storage, to return the robot arm to a position that makes the surgical instrument pass through the pivot position at a moving speed smaller than a moving speed of the robot arm moved using by the operation tool.

In the robotic surgical system according to the first aspect of the present disclosure, the controller performs return processing, if the surgical instrument is deviated from the pivot position stored in the storage, to return the robot arm to a position that makes the surgical instrument pass through the pivot position at a moving speed smaller than a moving speed of the robot arm moved using by the operation tool. Accordingly, the robot arm is returned to the position, which makes the surgical instrument pass through the pivot position, at a moving speed smaller than a moving speed of the robot arm moved through operation of the operation tool in the return processing. As a result, because the moving speed of the robot arm in a direction different from the direction in which an operator intends to move the robot arm is reduced, the operator is unlikely to perceive movement of the robot arm in the operator's unintended direction. Consequently, it is possible to prevent confusion of the operator caused by movement of the robot arm in a direction different from the operator's intended direction when the operator moves the robot arm through operation of an operation tool.

A surgical robot according to a second aspect of the present disclosure includes a robot arm having a distal end to which a surgical instrument is attached; a pivot-position setter that stores, in a storage, a pivot position on the surgical instrument attached to the robot arm that serves as a pivot point during movement; and a controller that performs return processing, if the surgical instrument is deviated from the pivot position stored in the storage, to return the robot arm to a position that makes the surgical instrument pass through the pivot position at a moving speed smaller than a moving speed of the robot arm moved by the operation tool.

In the surgical robot according to the second aspect of the present disclosure, the controller performs return processing, if the surgical instrument is deviated from the pivot position stored in the storage, to return the robot arm to a position that makes the surgical instrument pass through the pivot position at a moving speed smaller than a moving speed of the robot arm moved by the operation tool. Accordingly, the robot arm is returned to the position, which makes the surgical instrument pass through the pivot position, at a moving speed smaller than a moving speed of the robot arm moved through operation of the operation tool in the return processing. As a result, because the moving speed of the robot arm in a direction different from the direction in which an operator intends to move the robot arm is reduced, the operator is unlikely to perceive movement of the robot arm in the operator's unintended direction. Consequently, it is possible to provide a surgical robot capable of preventing confusion of the operator caused by movement of the robot arm in a direction different from the operator's intended direction when the operator moves the robot arm through operation of an operation tool.

A method for controlling a robotic surgical system according to a third aspect of the present disclosure includes storing, in a storage, a pivot position on a surgical instrument attached to a distal end of a robot arm that serves as a pivot point during movement; and returning the robot arm to a position that makes the surgical instrument pass through the pivot position at a moving speed smaller than a moving speed of the robot arm moved through operation of an operation tool that is operated to move the surgical instrument by using the robot arm if the surgical instrument is deviated from the pivot position stored in the storage.

In the method for controlling a robotic surgical system according to the third aspect of the present disclosure, as discussed above, the robot arm is returned to a position that makes the surgical instrument pass through the pivot position at a moving speed smaller than a moving speed of the robot arm moved through operation of an operation tool that is operated to move the surgical instrument by using the robot arm if the surgical instrument is deviated from the pivot position stored in the storage. Accordingly, the robot arm is returned to the position, which makes the surgical instrument pass through the pivot position, at a moving speed smaller than a moving speed of the robot arm moved through operation of the operation tool in the return processing. As a result, because the moving speed of the robot arm in a direction different from the direction in which an operator intends to move the robot arm is reduced, the operator is unlikely to perceive movement of the robot arm in the operator's unintended direction. Consequently, it is possible to provide a method for controlling a robotic surgical system capable of preventing confusion of the operator caused by movement of the robot arm in a direction different from the operator's intended direction when the operator moves the robot arm through operation of an operation tool.

A storage medium according to a fourth aspect of the present disclosure stores a program, the program including instructions for storing, in a storage, a pivot position on a surgical instrument attached to a distal end of a robot arm that serves as a pivot point during movement; and for returning the robot arm to a position that makes the surgical instrument pass through the pivot position at a moving speed smaller than a moving speed of the robot arm moved through operation of an operation tool that is operated to move the surgical instrument by using the robot arm if the surgical instrument is deviated from the pivot position stored in the storage.

The storage medium according to the fourth aspect of the present disclosure stores the program including the instruction for returning the robot arm to a position that makes the surgical instrument pass through the pivot position at a moving speed smaller than a moving speed of the robot arm moved through operation of an operation tool that is operated to move the surgical instrument by using the robot arm if the surgical instrument is deviated from the pivot position stored in the storage. Accordingly, the robot arm is returned to the position, which makes the surgical instrument pass through the pivot position, at a moving speed smaller than a moving speed of the robot arm moved through operation of the operation tool in the return processing. As a result, because the moving speed of the robot arm in a direction different from the direction in which an operator intends to move the robot arm is reduced, the operator is unlikely to perceive movement of the robot arm in the operator's unintended direction. Consequently, it is possible to provide a storage medium capable of preventing confusion of the operator caused by movement of the robot arm in a direction different from the operator's intended direction when the operator moves the robot arm through operation of an operation tool.

According to the present disclosure, it is possible to prevent confusion of an operator caused by movement of a robot arm in a direction different from the operator's intended direction when the operator moves the robot arm through operation of an operation tool.

### BRIEF DESCRIPTION OF THE DRAWINGS

FIG. 1 is a view showing a configuration of a robotic surgical system according to one embodiment.

FIG. 2 is a view showing a display of a medical cart according to the one embodiment.

FIG. 3 is a view showing a configuration of the medical cart according to the one embodiment.

FIG. 4 is a view diagram showing a configuration of a robot arm according to the one embodiment.

FIG. 5 is a view showing an instrument.

FIG. 6 is a perspective view showing a configuration of an arm operation unit according to the one embodiment.

FIG. 7 is a view illustrating translational movement of the robot arm.

FIG. 8 is a view illustrating rotational movement of the robot arm.

FIG. 9 is a view showing an endoscope.

FIG. 10 is a view showing a pivot-position setting tool.

FIG. 11 is a view showing an operation unit according to the one embodiment.

FIG. 12 is a view showing a right-hand side wrist part according to the one embodiment.

FIG. 13 is a view showing a left-hand side wrist part according to the one embodiment.

FIG. 14 is a perspective view showing foot pedals according to the one embodiment.

FIG. 15 is a control block diagram of the robotic surgical system according to the one embodiment.

FIG. 16 is a control block diagram of the robot arm according to the one embodiment.

FIG. 17 is a control block diagram of a positioner and the medical cart according to the one embodiment.

FIG. 18 is a control block diagram of the operation unit according to the one embodiment.

FIG. 19 is a view illustrating how to set a pivot position.

FIG. 20 is a flowchart illustrating a method for controlling the robotic surgical system according to the one embodiment.

FIG. 21 is a view showing rotational movement of the surgical instrument with the pivot position being set.

FIG. 22 is a view showing return of the deviated surgical instrument to make it pass through the pivot position.

FIG. 23 is a table illustrating insertion lengths of the surgical instrument and rotational moving amounts of the surgical instrument to cancel deviations of the surgical instrument.

FIG. 24 is a table illustrating turning amounts of the robot arm and degrees of moving speeds of the robot arm.

FIG. 25 is a flowchart illustrating return processing of the robotic surgical system according to the one embodiment.

### DESCRIPTION OF THE PREFERRED EMBODIMENT

### (Configuration of Robotic Surgical System)

The following description describes a configuration of a robotic surgical system 500 according to this embodiment. The robotic surgical system 500 includes a surgical robot 100, a remote operation apparatus 200, a vision unit 300 and an image processing unit 400.

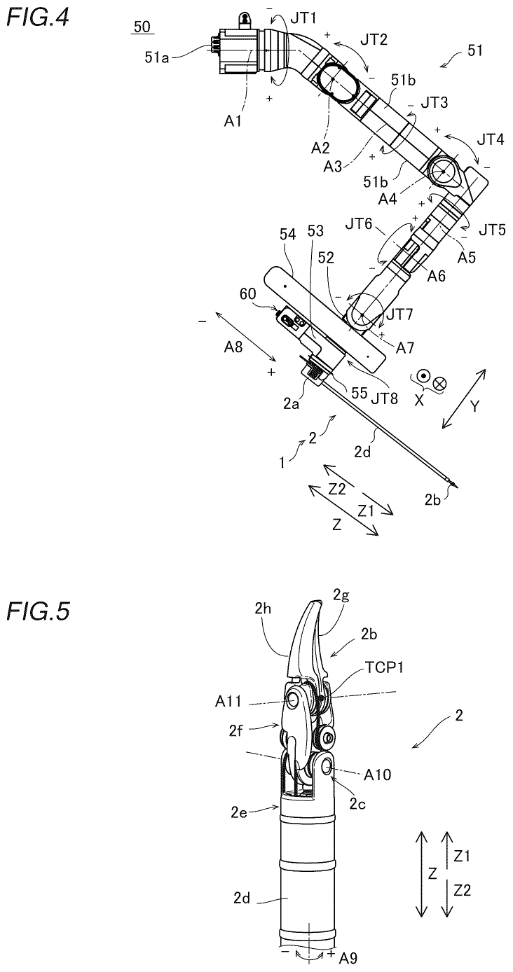

In this specification, a longitudinal direction of a surgical instrument 1 is defined as a Z direction as shown in FIG. 4. A distal end of the surgical instrument 1 is defined as a Z1 side, and a proximal end of the surgical instrument 1 is defined as a Z2 side. A direction perpendicular to the Z direction is defined as an X direction. A direction perpendicular to the Z direction and the X direction is defined as a Y direction.

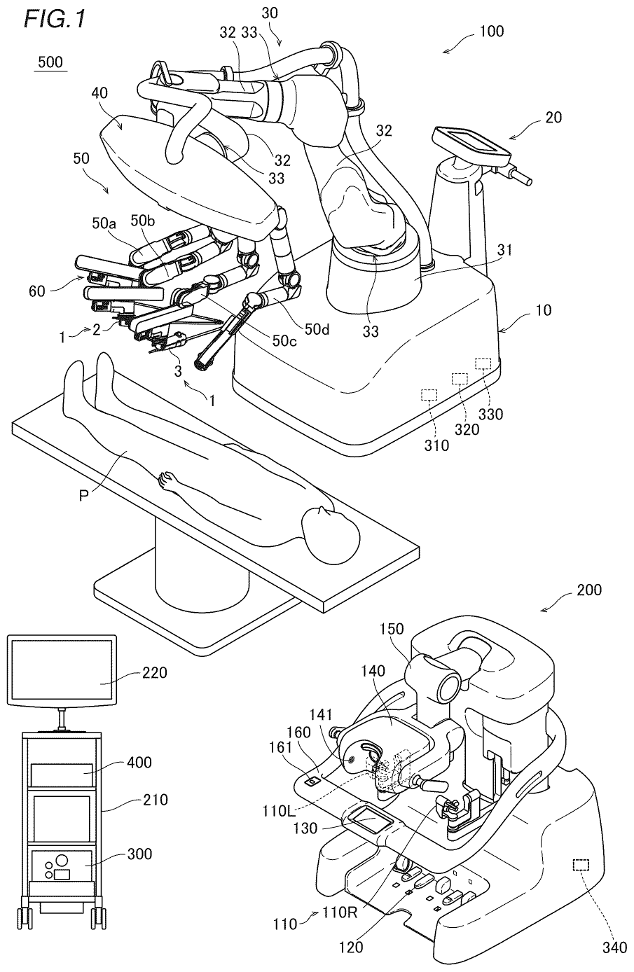

As shown in FIG. 1, the surgical robot 100 is arranged in an operating room. The remote operation apparatus 200 is located remote from the surgical robot 100. Also, the remote operation apparatus 200 receives instructions as to the surgical instruments 1. Specifically, an operator, such as a doctor, can provide the remote operation apparatus 200 with an instruction to instruct a desired motion of the surgical robot 100. The remote operation apparatus 200 transmits the provided command to the surgical robot 100. The surgical robot 100 performs the motion in accordance with the command received. The surgical robot 100 is arranged in the operating room, which is a sterile field.

### (Configuration of Surgical Robot)

As shown in FIG. 1, the surgical robot 100 includes a medical cart 10, a cart positioner operation unit 20, a positioner 30, an arm base 40, robot arms 50 and arm operation units 60 provided in the robot arms 50.

As shown in FIG. 3, the cart positioner operation unit 20 is arranged in a rear part of the medical cart 10 and supported by a cart positioner operation support 21, and the medical cart 10 or the positioner 30 can be moved in accordance with a manual operation of the cart positioner operation unit 20. The cart positioner operation unit 20 includes an input 22 and an operation handle 23. The input 22 is configured to accept instructions to move or change orientations of the positioner 30, the arm base 40 and the robot arms 50 to prepare a surgical operation mainly before the operation is carried out. The medical cart 10 includes the operation handle 23.

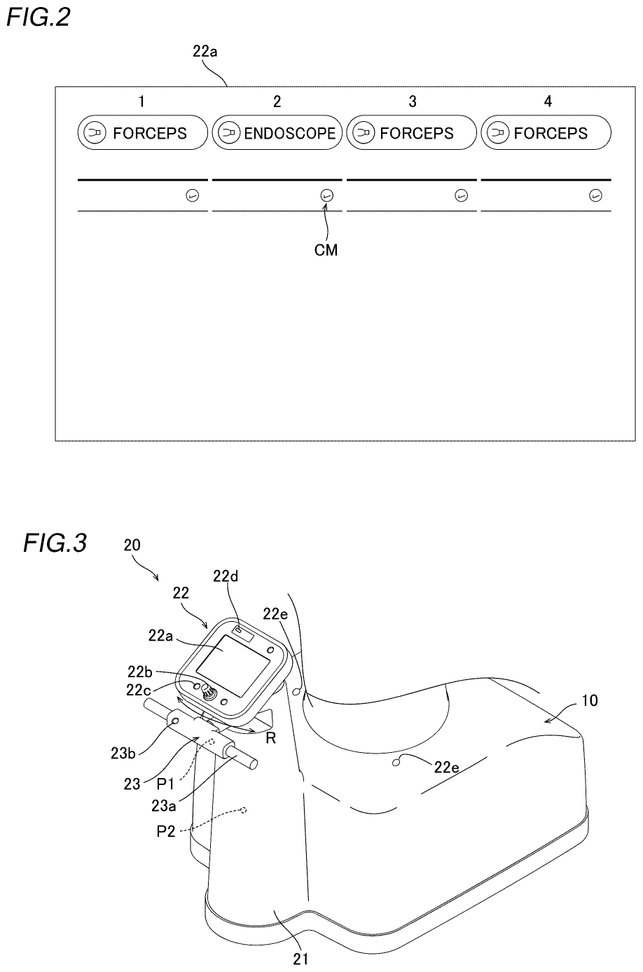

As shown in FIG. 3, the input 22 includes a display 22a, a joystick 22b, an enable switch 22c, an error reset button 22d and speakers 22e. For example, the display 22a is a liquid crystal panel. As shown in FIG. 2, the display 22a indicates numbers corresponding to the robot arms 50. Also, the display 22a indicates types of surgical instruments 1 attached to the robot arms 50. The display 22a indicates checkmarks CM representing that their pivot positions PP (discussed later) have been set.

As shown in FIG. 3, the joystick 22b is arranged in proximity to the display 22a of the input 22. When an operation mode displayed on the display 22a is selected, the positioner 30 can be three-dimensionally moved through operation of the joystick 22b.

The enable switch 22c is arranged in proximity to the joystick 22b. The enable switch 22c is configured to enable or disable movement of the positioner 30. When the enable switch 22c is pressed so that movement of the positioner 30 is enabled, the positioner 30 can be moved in accordance with a manual operation of the joystick 22b.

The error reset button 22d is configured to reset an error of the robotic surgical system 500. An exemplary error is an error of abnormal deviation. The speakers 22e are a pair of speakers. The pair of speakers 22e are arranged at a position in the medical cart 10 in proximity to the positioner 30.

Also, the operation handle 23 is arranged in proximity to the display 22a. The operation handle 23 includes a throttle grip 23a that is configured to be gripped and twisted by the operator, such as a nurse or engineer, to control movement of the medical cart 10. Specifically, the operation handle 23 is arranged under the input 22. The medical cart 10 can move forward when the throttle grip 23a is twisted from a near side toward a far side. The medical cart 10 can move backward when the throttle grip 23a is twisted from the far side toward the near side. A speed of the medical cart 10 can be changed in accordance with a twisting amount of the throttle grip 23a. In addition, the operation handle 23 is configured to swing leftward and rightward as shown by an R direction, and to rotate the medical cart 10 depending on the swinging operation of the operation handle 23.

Also, the operation handle 23 of the medical cart 10 includes an enable switch 23b configured to enable or disable movement. When the enable switch 23b is pressed so that movement of the medical cart 10 is enabled, the medical cart 10 can be moved in accordance with a manual operation of the throttle grip 23a of the operation handle 23.

For example, as shown in FIG. 1, the positioner 30 is constructed of a 7-axis multi-joint robot. The positioner 30 is arranged on the medical cart 10. The positioner 30 is configured to adjust a position of the arm base 40. The positioner 30 can three-dimensionally move the position of the arm base 40.

The positioner 30 includes a base 31, and links 32 coupled to the base 31. The links 32 are coupled to each other by joints 33.

The arm base 40 is attached to a distal end of the positioner 30. In the robot arms 50, the proximal end of each robot arm 50 is attached to the arm base 40. The robot arms 50 are foldable into a storage posture. The arm base 40 and the robot arms 50 are covered by sterile drapes when used. The robot arms 50 are configured to support the surgical instruments 1.

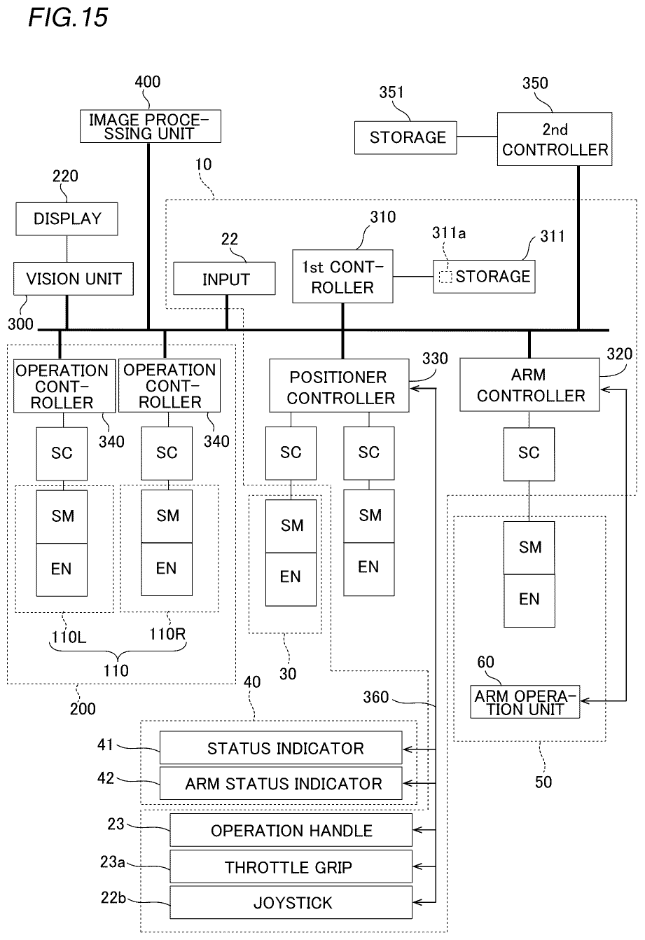

A status indicator 41 and an arm status indicator 42 shown in FIG. 15 are provided in the arm base 40. The status indicator 41 is configured to indicate a status of robotic surgical system 500. The arm status indicator 42 is configured to indicate states of the robot arms 50.

Two or more robot arms 50 are provided as the robot arms. Specifically, four robot arms 50a, 50b, 50c and 50d are provided. The robot arms 50a, 50b, 50c and 50d have a similar configuration to each other.

As shown in FIG. 4, each robot arm 50 includes an arm part 51, a first link part 52, a second link part 53, and a translation mechanism 54. The robot arm 50 includes joints JT1, JT2, JT3, JT4, JT5, JT6, JT7 and JT8. The joints JT1, JT2, JT3, JT4, JT5, JT6 and JT7 have A1, A2, A3, A4, A5, A6 and A7 axes as their rotation axes. JT8 has an A8 axis as its linear-motion axis. The arm part 51 includes a base section 51a and a link section 51b.

The arm part 51 is constructed of a 7-axis multi-joint robot arm. The first link part 52 is arranged in a distal end of the arm part 51. The arm operation unit 60 discussed later is attached to the second link part 53. The translation mechanism 54 is arranged between the first link part 52 and the second link part 53. The second link part 53 includes a holder 55 configured to hold the surgical instrument 1. The translation mechanism 54 is configured to translationally move the holder 55 to which the surgical instrument 1 is attached between a first position and a second position. The first position is a position of a Z2-direction side end of a moving range of the holder 55 moved by the translation mechanism 54 along the A8 axis. The second position is a position of a Z1-direction side end of the moving range of the holder 55 moved by the translation mechanism 54 along the A8 axis.

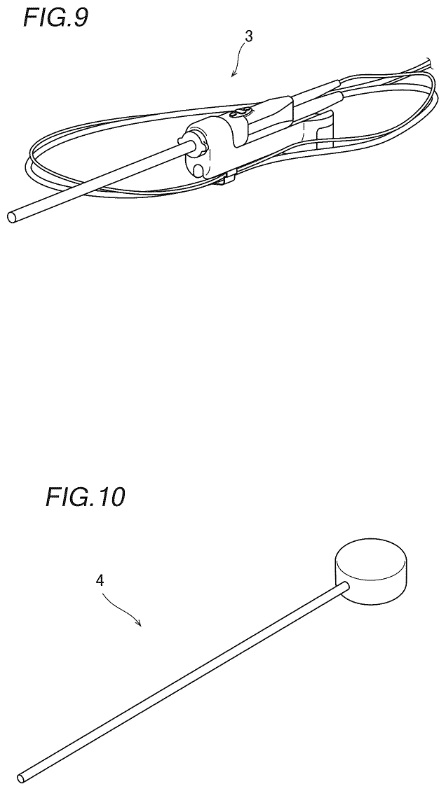

Surgical instruments 1 can be attached to the distal ends of the robot arms 50. The surgical instruments 1 include, for example, replaceable instruments 2, an endoscope 3 (see FIG. 9) configured to capture images of a part to be operated, a pivot-position setting tool 4 (see FIG. 10) to set a pivot position PP described below, and the like. The instrument 2 includes a driven unit 2a, an end effector 2b, a wrist joint 2c shown in FIG. 5, and a shaft 2d. The end effector 2b is connected to a distal end of the shaft 2d via the wrist joint 2c.

As shown in FIG. 1, the endoscope 3 is attached to the distal end of one, e.g., the robot arm 50c of the robot arms 50, and the instruments 2 are attached to the distal ends of the others, e.g., the robot arms 50a, 50b and 50d. The endoscope 3 is preferably attached to one of two robot arms 50b and 50c, which are located in a central part, of the four robot arms 50 arranged adjacent to each other.

### (Configuration of Instrument)

As shown in FIG. 5, the end effector 2b, which includes jaw members 2g and 2h for example, is attached to the distal end of the instrument 2. Scissors, a grasper, a needle holder, a microdissector, a staple applier, a tucker, a vacuum cleaning tool, a snare wire, a clip applier, or the like can be used as the end effector 2b.

The instrument 2 includes a first support member 2e and a second support member 2f. The first support member 2e is attached to the shaft 2d. The second support member 2f is rotatably supported by the first support member 2e about an A10 axis, and rotatably supports the end effector 2b about an A11 axis, which intersects the A10 axis. The shaft 2d rotates about an A9 axis. The wrist joint 2c is arranged between the second support member 2f and the first support member 2e, and is rotatable about the A10 axis as an axis of rotation.

### (Configuration of Arm Operation Unit)

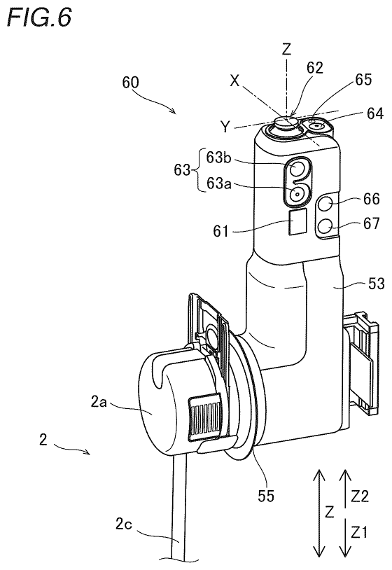

As shown in FIG. 6, the arm operation unit 60 is mounted to the robot arm 50, and is configured to operate the robot arm 50. Specifically, the arm operation unit 60 is mounted to the second link part 53.

The arm operation unit 60 includes an enable switch 61, a joystick 62, linear switches 63, a mode switching button 64, a mode indicator 65, a pivot button 66, and an adjustment button 67. The joystick 62 is an example of an operation tool. The pivot button 66 is an example of a pivot-position setter.

The enable switch 61 is configured to enable or disable movement of the robot arm 50, which is moved in accordance with an operation of the joystick 62 and the linear switches 63, when pressed. Movement of the surgical instrument 1 by the robot arm 50 is enabled when the enable switch 61 is pressed while the arm operation unit 60 is grasped by the operator, such as a nurse and assistant.

The joystick 62 is an operation tool that controls movement of the surgical instrument 1 by the robot arm 50. The joystick 62 controls a moving direction and a moving speed of the robot arm 50. The robot arm 50 can be moved in accordance with a tilting direction and a tilting angle of the joystick 62.

The linear switches 63 are switches for moving the surgical instrument 1 in the Z direction, which is a longitudinal direction of the surgical instrument 1. The linear switches 63 includes a linear switch 63a for moving the surgical instrument 1 in the direction in which the surgical instrument 1 is inserted into a patient P, and a linear switch 63b for moving the surgical instrument 1 in the direction in which the surgical instrument 1 is moved away from the patient P. The linear switch 63a and the linear switch 63b are constructed of press-button switches.

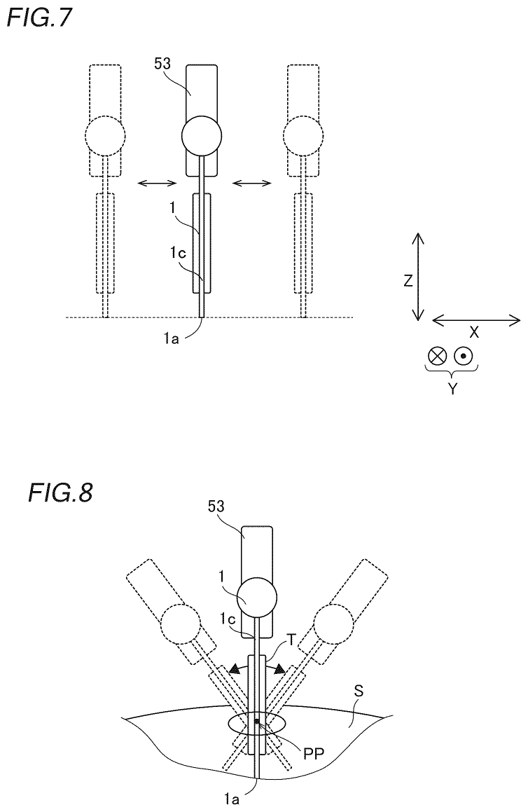

The mode switching button 64 is a press-button switch for switching between a translation mode in which the surgical instrument 1 is translationally moved, and a rotation mode in which the surgical instrument 1 is rotated. As shown in FIG. 7, in the translation mode in which the robot arm 50 is translationally moved, the robot arm 50 can be moved so that the distal end 1a of the surgical instrument 1 can be moved in an X-Y plane. As shown in FIG. 8, in the rotation mode in which the robot arm 50 is rotated, in a case in which any pivot position PP is not stored in the storage 351, the robot arm 50 can be moved so that the end effector 2b can be rotated about a center of the end effector 2b of the instrument 2 as the surgical instrument 1 on the A11 axis or the distal end of the end effector 2b as a pivot point, and in a case in which a pivot position PP is stored in the storage 351, the robot arm 50 can be moved so that the surgical instrument 1 can be rotated about the pivot position PP as a pivot point. In this case, the surgical instrument 1 is rotated with the shaft 1c of the surgical instrument 1 being inserted into a trocar T. The mode switching button 64 is arranged on a surface on a Z-direction side of the arm operation unit 60.

The mode indicator 65 is configured to indicate which mode is selected. The mode indicator 65 is configured to light on to indicate the rotation mode, and to light off to indicate the translation mode. The mode indicator 65 also serves as a pivot position indicator to indicate that the pivot position PP is set. The mode indicator 65 is arranged on the surface on the Z-direction side of the arm operation unit 60.

The pivot button 66 is a press-button switch configured to set the pivot position PP, which serves as the pivot point of the surgical instrument 1 attached to the robot arm 50. When the pivot button 66 is pressed, the pivot position PP is stored in the storage 351.

The adjustment button 67 is a button configured to optimize a position of the robot arm 50. After the pivot position PP is set with respect to the robot arm 50 to which the endoscope 3 is attached, positions of the other robot arms 50 and the arm base 40 are optimized when the adjustment button 67 is pressed. The adjustment button 67 is a button different from the enable switch 61.

(Remote Operation Apparatus) For example, as shown in FIG. 1, the remote operation apparatus 200 is arranged in an operating room or outside the operating room. The remote operation apparatus 200 includes operation units 110, foot pedals 120, a touch panel 130, a monitor 140, a support arm 150, a support bar 160, and an error reset button 161. The operation unit 110 serves as a handle for operation that receives commands from the operator, such as a doctor.

### (Operation Unit)

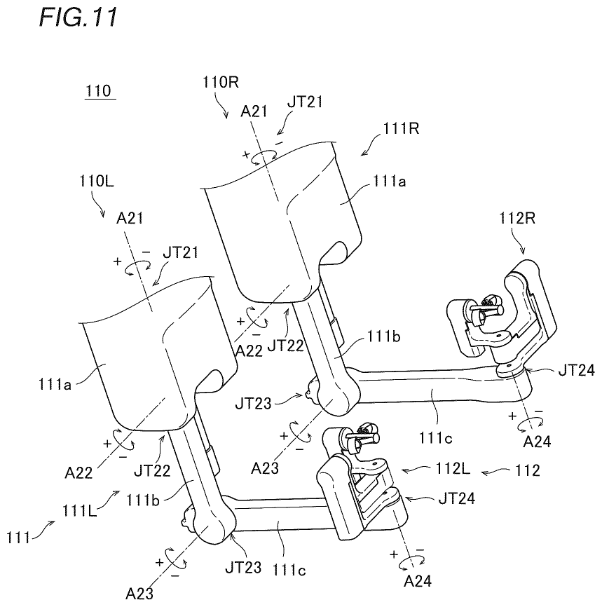

As shown in FIG. 11, the operation unit 110 is the handle configured to operate the surgical instruments 1. Also, the operation unit 110 receives operation instructions for the surgical instruments 1. The operation units 110 include an operation unit 110L that is arranged on a left side from viewpoint of the operator such as a doctor and is configured to be manually operated by the operator's left hand, and an operation unit 11CR that is arranged on a right side from viewpoint of the operator and is configured to be manually operated by the operator's right hand. The operation units 110 include arm parts 111 and wrist parts 112. The operation unit 11CR includes an arm part 111R and a wrist part 112R. The operation unit 110L includes an arm part 111L and a wrist part 112L.

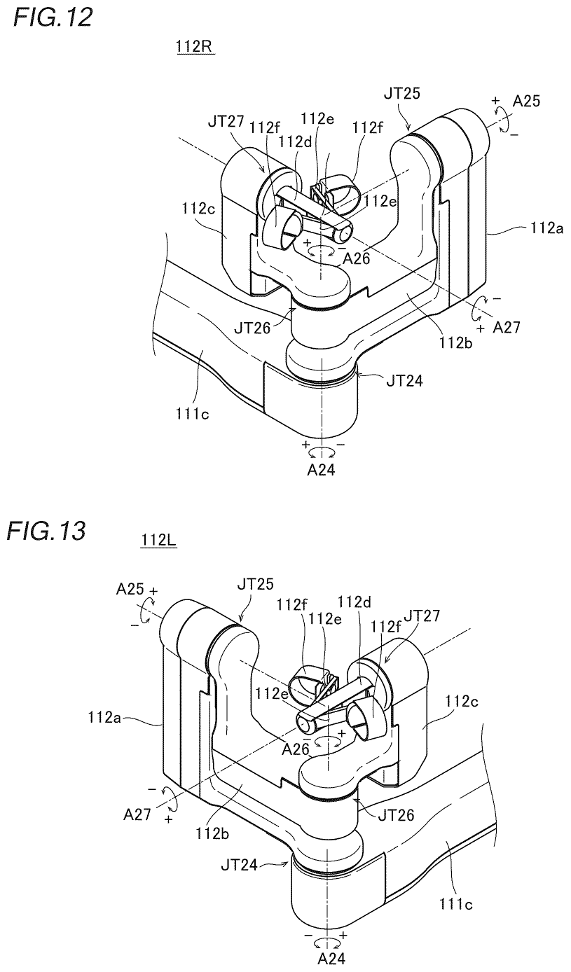

The arm parts 111 include joints JT21, JT22 and JT23 shown in FIG. 11, and joints JT24, JT25, JT26 and JT27 shown in FIGS. 12 and 13. The joints JT21, JT22, JT23, JT24, JT25, JT26 and JT27 have axes A21, A22, A23, A24, A25, A26 and A27.

### (Arm Part)

As shown in FIG. 11, the arm part 111R includes a link 111a, a link 1l1b and a link 1l1c. An upper end side of the link 111a is attached to the remote operation apparatus 200 rotatably about the A21 axis extending in a vertical direction. An upper end side of the link 1l1b is attached to a lower part of the link 111a rotatably about the A22 axis extending in a horizontal direction. One end side of the link 1l1c is attached to a lower end side of the link 1l1b rotatably about the A23 axis extending in a horizontal direction. The wrist part 112 is attached to another end side of the link 1l1c rotatably about the A24 axis. The link 111a is connected to the remote operation apparatus 200 by the joint JT21. The link 111a is connected to the link 1l1b by the joint JT22. The link 1l1b is connected to the link 1l1c by the joint JT23. The arm part 111 supports the wrist part 112. Here, the arm part 111L has a configuration similar to the arm part 111R.

The wrist parts 112 include a wrist part 112R shown in FIG. 12 operated by the operator's right hand, and a wrist part 112L shown in FIG. 13 operated by the operator's left hand. A reference posture of the operation unit 110R is shown in FIG. 12, and a reference posture of the operation unit 110L is shown in FIG. 13. A configuration of the wrist part 112R is similar to the wrist part 112L.

The wrist part 112 includes a link 112a, a link 112b, a link 112c, and a grip support 112d that is operated by the operator (e.g., a doctor). The link 112a includes a proximal end connected to a distal end of the arm part 111, and is configured to rotate about the A24 axis. The link 112b includes a proximal end connected to a distal end of the link 112a with respect to link 112a, and is configured to rotate about the A25 axis. The link 112c includes a proximal end connected to a distal end of the link 112b and a distal end to which the grip support 112d is connected, and is configured to rotate relative to the link 112b about the A26 axis. The grip support 112d rotates about the A27 axis relative to the link 112c. The link 112a, the link 112b and the link 112c have L shapes.

Each wrist part 112 includes a pair of grips 112e operated by the operator to be opened and closed. The grips 112e are formed of thin plate-shaped levers, and near-side ends of the pair of grips 112e are rotatably coupled to a near-side end of the grip support 112d. The grips 112e include cylindrical finger insertion sections 112f. The operator can insert his or her fingers into the finger insertion sections 112f, and operate the wrist part 112. Proximal ends of the pair of grips 112e are coupled to the grip support 112d so that an opening angle between the jaw 2g and the jaw 2h can be changed by increasing/decreasing an angle between the pair of grips 112e. One of the grips 112e includes a magnet, while the grip support 112d includes a Hall sensor. The magnet and the Hall sensor function as an angle detection sensor, and can output the opening angle when the operator opens/closes the grips 112e. Here, one of the grips 112e may include a Hall sensor, while the grip support 112d may include a magnet so that they form the angle detection sensor.

As shown in FIG. 1, the monitor 140 is a scope-type display device configured to display an image captured by the endoscope 3. The monitor 140 includes an information producer 141. The information producer 141 is configured to produce an error sound. The support arm 150 supports the monitor 140, and can adjust a height of the monitor 140 to a height of eyes of the operator such as a doctor. The touch panel 130 is arranged on the support bar 160. When the operator's head is detected by a sensor arranged in proximity to the monitor 140, the surgical robot 100 can accept manual operations from the remote operation apparatus 200. The operator manually operates the operation unit 110 and the foot pedals 120 while viewing an affected area on the monitor 140. Commands can be input to the remote operation apparatus 200 in accordance with these manual operations. Commands input to the remote operation apparatus 200 are transmitted to the surgical robot 100.

### (Foot Pedals)

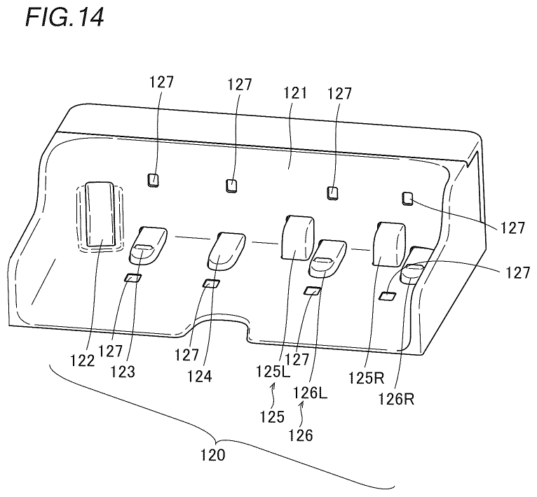

As shown in FIG. 14, the foot pedals 120 are configured to activate functions of the surgical instruments 1. The foot pedals 120 are provided in a base 121. The foot pedals 120 include a switching pedal 122, a clutch pedal 123, a camera pedal 124, incision pedals 125, coagulation pedals 126, and foot detectors 127. The switching pedal 122, the clutch pedal 123, the camera pedal 124, the incision pedals 125, the coagulation pedals 126 are operated by the operator's foot. Also, the incision pedals 125 include an incision pedal 125R corresponding to a right-side robot arm 50 and an incision pedal 125L corresponding to a left-side robot arm 50. Also, the coagulation pedals 126 include a coagulation pedal 126R corresponding to a right-side robot arm 50 and a coagulation pedal 126L corresponding to a left-side robot arm 50.

The switching pedal 122 is configured to switch between the robot arms 50 to be operated by the operation unit 110. The clutch pedal 123 is configured to activate a clutch function of temporally halting operation connection between the robot arm 50 and the operation unit 110. While the clutch pedal 123 is pressed by the operator, instructions provided by the operation unit 110 are not transmitted to the robot arm 50. While the camera pedal 124 is pressed by the operator, the robot arm 50 that holds the endoscope 3 can be operated through the operation unit 110. While the incision pedal 125 or the coagulation pedal 126 is pressed, an electric surgical apparatus is active.

### (Vision Unit and Image Processing Unit)

As shown in FIG. 1, a cart 210 holds a vision unit 300 and an image processing unit 400. The image processing unit 400 is configured to process an image captured by the endoscope 3. A display 220 is arranged on the cart 210. The display 220 is configured to display the image captured by the endoscope 3.

### (Configuration of Control System)

As shown in FIG. 15, the robotic surgical system 500 includes a first controller 310, an arm controller 320, a positioner controller 330, operation controllers 340 and a second controller 350. In addition, the robotic surgical system 500 includes a storage 311 connected to the first controller 310, and a storage 351 connected to the second controller 350. The first controller 310 is an example of a controller.

The first controller 310 is accommodated in the medical cart 10, and configured to communicate with the arm controller 320 and the positioner controller 330 so that the robotic surgical system 500 is entirely controlled. Specifically, the first controller 310 controls the arm controller 320, the positioner controller 330 and the operation controllers 340 by using the communications with them. The first controller 310 is connected to the arm controller 320, the positioner controller 330 and the operation controllers 340 through LAN, or the like. The first controller 310 is arranged in the medical cart 10.

Each of the robot arms 50 includes the arm controller 320. In other words, arm controllers 320 the number of which corresponds to the number of the robot arms 50 are included in the medical cart 10.

As shown in FIG. 15, the input 22 is connected to the first controller 310 through LAN, or the like. The status indicator 41, the arm status indicator 42, the operation handle 23, the throttle grip 23a, and the joystick 22b are connected to the positioner controller 330 through a wiring line 360 by means of a communication network that can share information with them using serial communication. Although all of the status indicator 41, arm status indicator 42, and the like are connected to each other through one wiring line 360 in FIG. 15, wiring lines 360 are actually provided to each of the status indicator 41, the arm status indicator 42, the operation handle 23, the throttle grip 23a, the joystick 22b, the stabilizer 24 and the electric cylinder 25.

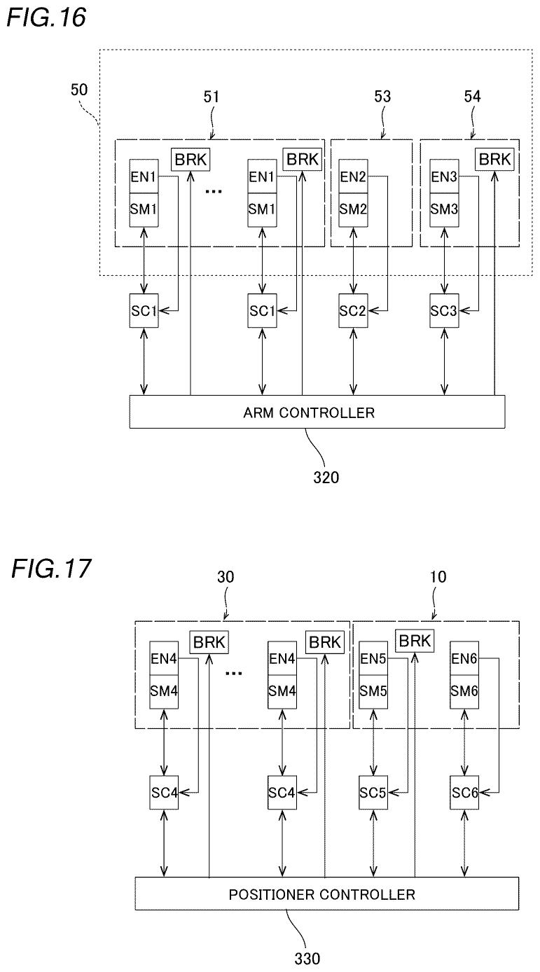

As shown in FIG. 16, the arm part 51 includes servomotors SM1, encoders EN1 and speed reducers corresponding to the joints JT1, JT2, JT3, JT4, JT5, JT6 and JT7. The encoder EN1 is configured to detect a rotation angle of the servomotor SM1. The speed reducer is configured to reduce a rotation of the servomotor SM1 whereby increasing its torque. A servo controller SC1 controls the servomotor SM1, and is arranged in the medical cart 10 adjacent to the arm controller 320. Also, the encoder EN1 is configured to detect the rotation angle of the servomotor SM1, and is electrically connected to the servo controller SC1.

The second link part 53 includes a servomotor SM2 configured to rotate a driven member arranged in a driven unit 2a of the surgical instrument 1, an encoder EN2, and a speed reducer. The encoder EN2 is configured to detect a rotation angle of the servomotor SM2. The speed reducer is configured to reduce a rotation of the servomotor SM2 whereby increasing its torque. The medical cart 10 includes a servo controller SC2 that controls the servomotor SM2 for driving the surgical instrument 1. The encoder EN2 for detecting the rotation angle of the servomotor SM2 is electrically connected to the servo controller SC2. Here, such servomotors SM2, such encoders EN2 and such servo controllers SC2 are included.

The translation mechanism 54 includes a servomotor SM3 configured to translationally move the surgical instrument 1, an encoder EN3, and a speed reducer. The encoder EN3 is configured to detect a rotation angle of the servomotor SM3. The speed reducer is configured to reduce a rotation of the servomotor SM3 whereby increasing its torque. The medical cart 10 includes a servo controller SC3 that controls the servomotor SM3 for translationally moving the surgical instrument 1. The encoder EN3 for detecting the rotation angle of the servomotor SM3 is electrically connected to the servo controller SC3.

The first controller 310 is configured to generate instruction values that specify positions of the servomotor SM1, SM2 and SM3 in accordance with manual operation that is received by the remote operation apparatus 200, and to drive the servomotor SM1, SM2 and SM3 in accordance with the instruction values. If any of differences between instruction values and positions of the servomotor SM1, SM2 and SM3 detected by sensors becomes greater than an allowable range, the first controller 310 determines an error of abnormal deviation.

As shown in FIG. 17, the positioner 30 includes servomotors SM4, encoders EN4 and speed reducers corresponding to joints 33 of the positioner 30. Each encoder EN4 is configured to detect a rotation angle of the servomotor SM4. The speed reducer is configured to reduce a rotation of the servomotor SM4 whereby increasing its torque.

The medical cart 10 includes wheels including front wheels as driving wheels, and rear wheels configured to be steered by manually operating the operation handle 23. The rear wheels are arranged closer to the operation handle 23 with respect to the front wheels. The medical cart 10 includes a servomotor SM5 configured to drive the front wheels of the medical cart 10, an encoder EN5, speed reducers, and brakes BRK. The speed reducer is configured to reduce a rotation of the servomotor SM5 whereby increasing its torque. Also, the operation handle 23 includes a potentiometer P1 shown in FIG. 3, and the servomotor SM5 of the front wheels can be driven in accordance with a rotation angle detected by the potentiometer P1 in response to a twisting amount of the throttle grip 23a. The rear wheels of the medical cart 10 have a twin-wheel type structure, and the rear wheels can be steered in accordance with a rightward/leftward turn of the operation handle 23. Also, the operation handle 23 includes a potentiometer P2 shown in FIG. 3 on a turning shaft, and the rear wheel of the medical cart 10 is provided with a servomotor SM6, an encoder EN6, and speed reducers. The speed reducer is configured to reduce a rotation of the servomotor SM6 whereby increasing its torque. The servomotor SM6 can be driven in accordance with a rotation angle detected by the potentiometer P2 in response to a rightward/leftward turning amount of the operation handle 23. In other words, power is assisted by the servomotor SM6 when the rear wheels are steered by turning the operation handle 23 rightward or leftward.

The medical cart 10 can be moved forward or rearward by driving the front wheels. Also, the medical cart 10 can be turned rightward or leftward by steering the rear wheels by turning the operation handle 23.

As shown in FIG. 17, the medical cart 10 includes servo controllers SC4 that controls the servomotors SM4 for moving the positioner 30. Also, the encoder EN4 is configured to detect the rotation angle of the servomotor SM4, and is electrically connected to the servo controller SC4. The medical cart 10 includes a servo controller SC5 that controls the servomotor SM5 for driving the front wheels of the medical cart 10. The encoder EN5 for detecting the rotation angle of the servomotor SM5 is electrically connected to the servo controller SC5. The medical cart 10 includes a servo controller SC6 that controls the servomotor SM6 for power assistance to steering of the rear wheels of the medical cart 10. The encoder EN6 for detecting the rotation angle of the servomotor SM6 is electrically connected to the servo controller SC6.

As shown in FIGS. 16 and 17, the joints JT1, JT2, JT3, JT4, JT5, JT6 and JT7 of the arm part 51, and the joints 33 of the positioner 30 include their brakes BRK. Also, the front wheels of the medical cart 10, the arm base 40 and the translation mechanism 54 include their brakes BRK. The arm controller 320 is configured to one-directionally transmit control signals to the brakes BRK of the joints JT1, JT2, JT3, JT4, JT5, JT6 and JT7 of the arm part 51, and the translation mechanism 54. The control signals indicate on/off of the brakes BRK. The signals indicating on of the brakes BRK include a signal that instructs the brake BRK to keep activating. The control signals transmitted from the positioner controller 330 to the brakes BRK included in the joints 33 of the positioner 30 and the arm base 40 are similar to the control signals transmitted from the arm controller. On startup, all the brakes BRK of the arm base 40, the arm part 51 and the translation mechanism 54 are turned off but the servomotors SM are driven to keep postures of the robot arm 50 and the arm base 40 against gravity. If an error occurs in the robotic surgical system 500, the brakes BRK included in the arm base 40, the arm part 51 and the translation mechanism 54 are turned on. When the error in the robotic surgical system 500 is reset, the brakes BRK included in the arm base 40, the arm part 51 and the translation mechanism 54 are turned off. When shutdown operation is performed in the robotic surgical system 500, the brakes BRK included in the arm base 40, the arm part 51 and the translation mechanism 54 are turned on. The brakes BRK of the front wheels of the medical cart 10 are constantly turned on, and the brakes BRK are deactivated only when the enable switch 23b is kept pressed. Also, the brakes BRK of the joints 33 of the positioner 30 are constantly turned on, and the brakes BRK are deactivated only when the enable switch 22c is kept pressed.

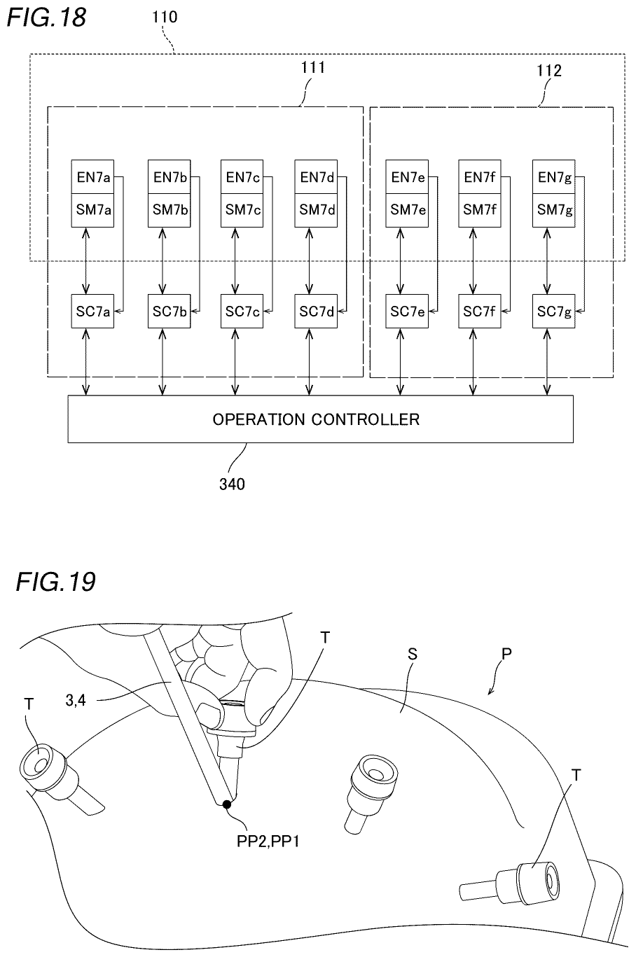

As shown in FIG. 18, the joints JT21, JT22, JT23, JT24, JT25, JT26 and JT27 of the operation unit 110 includes servomotors SM7a, SM7b, SM7c, SM7d, SM7e, SM7f and SM7g, respectively. The servomotor SM7a rotates the link 111a about the A21 axis. The servomotor SM7b rotates the link 1l1b about the A22 axis. The servomotor SM7c rotates the link 1l1c about the A23 axis. The servomotor SM7d rotates the link 112a about the A24 axis. The servomotor SM7e rotates the link 112b about the A25 axis. The servomotor SM7f rotates the link 112c about the A26 axis. The servomotor SM7g rotates the grip support 112d about the A27 axis. Also, servo controllers SC7a, SC7b, SC7c, SC7d, SC7e, SC7f and SC7g that control the servomotors are provided. Encoders EN7a, EN7b, EN7c, EN7d, EN7e, EN7f and EN7g for detecting rotation angles of the servomotors are electrically connected to the servo controllers. Each of the operation units 110L and 11CR includes the servomotors, the servo controllers and the encoders.

The first controller 310 controls the servomotors through the operation controllers 340 so that torques are produced to cancel out gravitational torques applied to the rotation axes of the servomotors in postures of the operation units 110. Accordingly, the operator can manually operate the operation units 110 by relatively small forces.

When the operator rotationally operates the grip support 112d about the A27 axis of the operation unit 110 shown in FIGS. 12 and 13, the shaft 2d of the instrument 2 rotates about the A9 axis shown in FIG. 5. When the operator rotationally operates the joints JT24, JT25, and JT26 of the operation unit 110 shown in FIGS. 12 and 13, the end effector 2b pivots about the A10 axis or the A11 axis shown in FIG. 5.

The operation controllers 340 are provided in a main body of the remote operation apparatus 200. The operation controllers 340 control the operation units 110. The operation controllers 340 are associated with both the left-hand side operation unit 110L and the right-hand side operation unit 11CR as shown in FIG. 15.

As shown in FIG. 15, the vision unit 300 and the image processing unit 400 are connected to the first controller 310 through LAN. The display 220 is connected to the vision unit 300.

(Setting of Pivot Position) Setting of the pivot position PP is now described. As shown in FIG. 19, the operator first moves the robot arm 50 through operation of the arm operation unit 60 to move the distal end of the endoscope 3, which is attached to the distal end side of the robot arm 50, to a position corresponding to an insertion position of the trocar T inserted through a body surface S into a body of a patient P, and then operates the pivot button 66 so that the second controller 350 stores the pivot position PP2 of the endoscope 3 into the storage 351. Similarly, the operator first moves the robot arm 50 to move the distal end of the pivot-position setting tool 4, which is attached to the distal end side of the robot arm 50, to a position corresponding to an insertion position of the trocar T inserted through the body surface S into the body of the patient P, and then operates the pivot button 66 so that the second controller 350 stores the pivot position PP1 of the instrument 2 into the storage 351. Here, operating the pivot button 66 means pressing the pivot button 66. The pivot position PP1 and the pivot position PP2 are collectively referred to as pivot positions PP.

### (Control Function of First Controller)

The following description describes control by the first controller 310 when the surgical instrument 1 is deviated from the pivot position PP stored in the storage 351. Here, the pivot position PP has been already stored in the storage 351. The surgical robot 100 stops when the robot arm 50 interferes with other components or the operator. The control described below is executed when the surgical robot 100 is restarted after being stopped. In addition, the control described below is executed when the surgical instrument 1 is positioned inside the body of the patient P. The control described below is executed when the surgical instrument 1 is either the instrument 2 or the endoscope 3. The control described below is also executed for each of the four robot arms 50.

As shown in FIG. 15, a program 311a, which includes processes of step S1 to S3 described below, is stored in the storage 311. The storage 311 is an example of a storage medium. The program 311a may be stored in the storage 351.

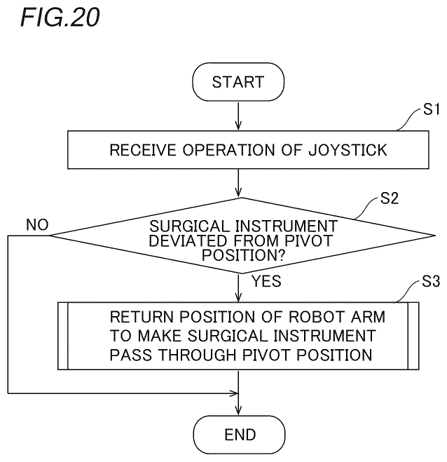

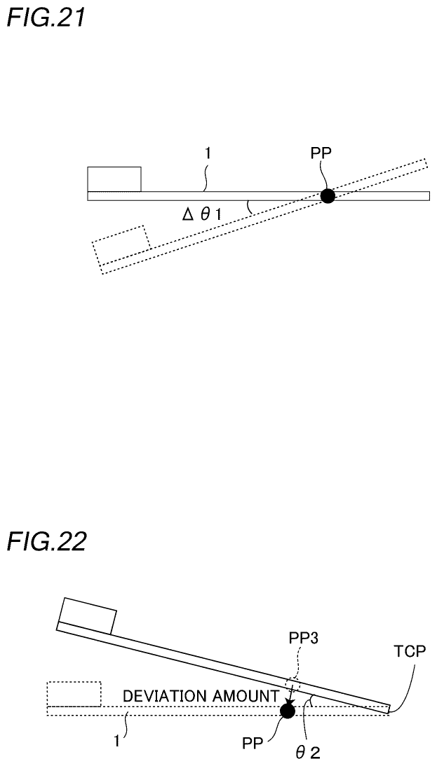

In step S1 shown in FIG. 20, the operator operates the joystick 62 of the arm operation unit 60. Correspondingly, the first controller 310 receives the operation of the movement of the surgical instrument 1 by the robot arm 50. As shown in FIG. 21, the robot arm 50 is moved to rotate the surgical instrument 1 about the pivot position PP, which serves as the pivot point, if the pivot position PP is stored in the storage 351. Where the rotational moving amount of the surgical instrument 1 is defined as Δθ1 [deg/cycle], Δθ1 is represented by the equation shown below. Here, [deg/cycle] is a rotational moving amount per control cycle. The rotational moving amount per control cycle means the moving speed. Here, the control cycle is 4 ms, for example.

$$Δθ1 = operation amount of joystick 62 \times moving speed of robot arm \\ \\ 50 \times C 1.$$

C1 is a constant and is set so that the operator can operate the joystick 62 without feeling that something is wrong when moving the robot arm 50 using the joystick, for example. C1 is 0.006×0.5, for example.

The operation amount of the joystick 62 is a dimensionless quantity, and is a value from −10 to +10, for example. The operation amount of the joystick 62 corresponds to the inclination angle of the joystick 62. Return processing described later is performed to return the position of the robot arm 50 to within the ranges of operation amounts of the joystick 62 of −10 to −5 and +5 to +10. The return processing may be performed within the entire range of operation amounts of the joystick 62.

In this embodiment, the degree of moving speed of the robot arm 50 is received via the display 22a of the input 22. For example, five degrees of moving speed, which are speed 1, speed 2, speed 3, speed 4 and speed 5, are prepared. The display 22a of the input 22 is a touch panel, and the operator sets the degree of moving speed by pressing the touch panel, for example. The degrees of moving speed are speeds 1, 2, 4 and 5, which are defined as 0.5, 0.75, 1.25 and 1.5, respectively, while speed 3 is defined as 1.0. Specifically, values [deg/s] of speeds 1, 2, 3, 4 and 5 are 3.75, 5.625, 7.5, 9.375 and 11.25, respectively, for example. These values are the same irrespective of whether the pivot position PP is set or not. Here, these values are merely illustrative, and values [deg/s] of speeds are not limited to these examples. The display 22a of the input 22 is an example of a setting receiver.

In step S2 shown in FIG. 20, the first controller 310 determines whether the surgical instrument 1 is deviated from the pivot position PP stored in the storage 351. For example, the first controller 310 acquires the coordinates of the surgical instrument 1 at the time of restart based on information from the encoders EN1, EN2 and EN3 arranged at the joints JT1 to JT8 of the robot arm 50. The first controller 310 reads the coordinates of the pivot position PP from the storage 351, and if the deviation between the coordinates of the read pivot position PP and the coordinates of the surgical instrument 1 acquired after the restart is greater than a predetermined threshold, the first controller 310 determines that the surgical instrument 1 is deviated from the coordinates of the pivot position PP stored in the storage 351. Here, the predetermined threshold is a value close to zero, for example.

If determining that the surgical instrument 1 is deviated from the pivot position PP stored in the storage 351, the first controller 310 performs the return processing to return the position of the robot arm 50 at a moving speed smaller than the moving speed of the robot arm 50 moved through operation of the joystick 62 so that the surgical instrument 1 passes through the pivot position PP in step S3. Specifically, as shown in FIG. 22, the normal to the shaft 2d of the surgical instrument 1 is drawn from the pivot position PP so that the intersection of the normal and the shaft 2d is set as a provisional pivot position PP3, and the robot arm 50 is then slowly returned to a position that makes the provisional pivot position PP3 coincide with the pivot position PP stored in the storage 351. In this embodiment, the first controller 310 performs the return processing at a moving speed smaller than the moving speed of the robot arm 50 that is moved through operation of the joystick 62. Here, in step S2, if determining that the surgical instrument 1 is not deviated from the pivot position PP stored in the storage 351, the first controller 310 does not perform the return processing. Specifically, the return processing is described below.

### (Return Processing)

The moving speed of the robot arm 50 is based on the degree of moving speed set via the display 22a of the input 22. Here, when the rotational moving amount of the surgical instrument 1 is Δθ2 [deg/cycle], the rotational moving amount of the surgical instrument 1 in the return processing Δθ2 is represented by the following equation:

Δθ2=arctan (deviation amount of pivot position PP/insertion length of surgical instrument 1). Here, as shown in FIG. 22, the deviation amount of the pivot position PP is a distance between the provisional pivot position PP3 and the pivot position PP stored in the storage 351. The insertion length of the surgical instrument 1 is a length between the pivot position PP and the distal end or Tool Center Point (TCP) of the surgical instrument 1. Although the surgical instrument 1 is illustrated to rotate about TCP in FIG. 22, when the robot arm 50 is moved through operation of the joystick 62, the deviated surgical instrument 1 is returned to make it pass through the pivot position PP while TCP is moved.

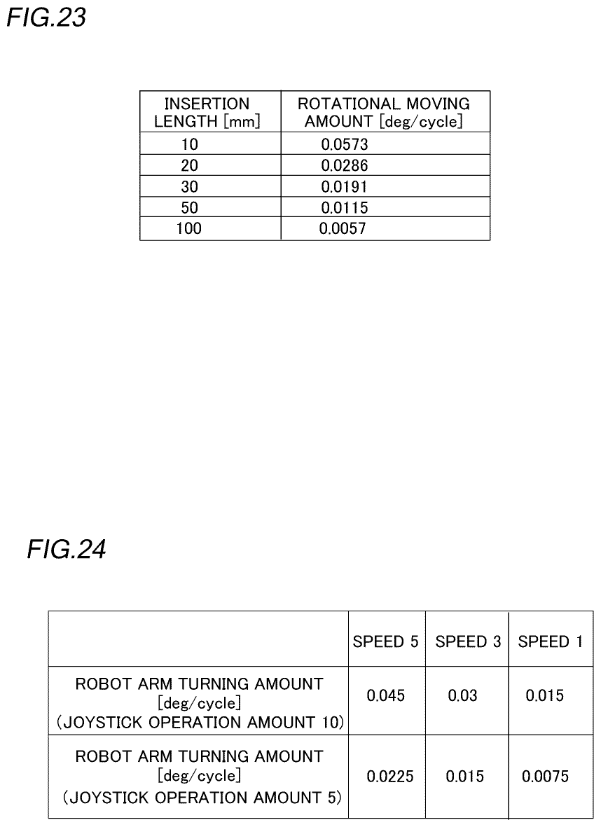

Here, the return processing that is performed without restriction on the moving speed is described. As shown in FIG. 23, the rotational moving amount of the surgical instrument 1 varies depending on the insertion length of the surgical instrument 1, even when the provisional pivot position PP3 is moved by an identical amount to return to the pivot position PP stored in the storage 251. For example, as shown in FIG. 23, the rotational moving amounts [deg/cycle] of the surgical instrument 1 are 0.0573, 0.2286, 0.0191, 0.0115 and 0.0057 when the insertion lengths are 10 mm, 20 mm, 30 mm, 50 mm and 100 mm, respectively. Here, these values are merely illustrative, and values [deg/s] of rotational moving amounts are not limited to these examples. In other words, the smaller the insertion length, the larger the rotational moving amount. As shown in FIG. 24, the turning amount of the robot arm 50, which is turned through operation of the joystick 62, varies depending on the degree of moving speed set by using the display 22a of the input 22. The turning amount of the robot arm 50 is an amount of rotation about the pivot position PP. For example, the turning amounts [deg/cycle] of the robot arm 50 are 0.045, 0.03 and 0.015, when the set degrees of moving speed are speed 5, speed 3, and speed 1, respectively, in the case in which the operation amount of the joystick 62 is 10. Also, the turning amounts [deg/cycle] of the robot arm 50 are 0.0225, 0.015 and 0.0075, when the set degrees of moving speed are speed 5, speed 3, and speed 1, respectively, in the case in which the operation amount of the joystick 62 is 5. Here, these values are merely illustrative and turning amounts are not limited to these examples. In other words, even in the case in which the degree of moving speed is speed 5, when the insertion length is 10 mm, the rotational moving amount of the surgical instrument 1 is greater than the turning amount of the robot arm 50 by the joystick 62. In other words, if the direction in which the operator attempts to move the robot arm 50 differs from the direction in which the deviated surgical instrument 1 is returned to make it pass through the pivot position PP, the operator may perceive that the robot arm 50 moves in a different direction than the operator's intended direction.

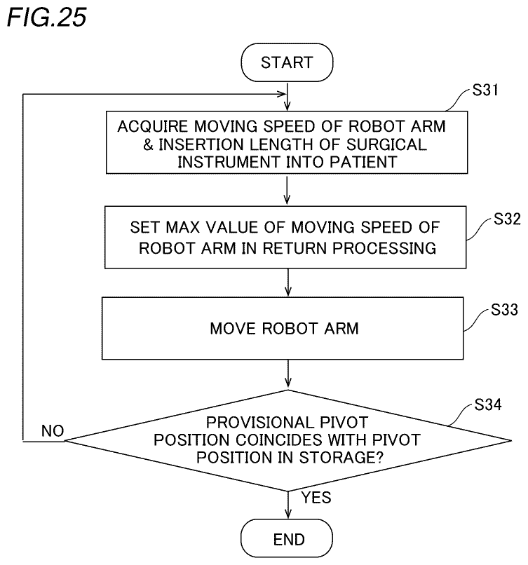

To address this, in step S31 shown in FIG. 25, the first controller 310 acquires the moving speed of the robot arm 50, which is moved through operation of the joystick 62, and the insertion length of the surgical instrument 1 inserted into the patient P. The moving speed of the robot arm 50 is the value that is received via the display 22a of the input 22. The insertion length is acquired based on information from the encoders EN1, EN2 and EN3 arranged at the joints JT1 to JT8 of the robot arm 50.

Subsequently, in step S32, in this embodiment, the first controller 310 sets the maximum value of the moving speed of the robot arm 50 in the return processing based on the moving speed of the robot arm 50 moved through operation of the joystick 62 and the insertion length of the surgical instrument 1 inserted into the patient P. Firstly, a turning amount Δθ3 [deg/cycle] in the return processing is represented by the following equation:

Δθ3=operation amount of joystick 62×moving speed of robot arm 50×C2, where C2 is a constant, e.g., 0.006×0.5 similar to C1. Also, the maximum value of the moving speed [mm/cycle] in the return processing is then represented by the following equation:

$$maximum value of moving speed = tan ( Δθ3 ) \times insertion length .$$

Subsequently, in step S33, the robot arm 50 is moved so that the surgical instrument 1 passes through the pivot position PP. Because the maximum value of the moving speed is set as described above, the robot arm 50 is moved at a moving speed smaller than the moving speed of the robot arm 50, which is moved through operation of the joystick 62, so that the surgical instrument 1 passes through the pivot position PP.

In this embodiment, the first controller 310 gradually increases the moving speed in the return processing. For example, the moving speed at the start of the return processing is 0.00002 mm/cycle. The moving speed increases by 0.00002 mm/cycle with each control cycle. Here, these values are merely illustrative and the above moving speed and moving speed increasing amount are not limited to these examples.

In addition, in this embodiment, the upper limit of the maximum value of the moving speed is previously set. For example, the upper limit of the maximum value of the moving speed is 0.01 mm/cycle. That is, the maximum value of the moving speed does not exceed 0.01 mm/cycle even when the value represented by the above equation increases. Here, this value is merely illustrative, and the upper limit is not limited to this example. If the moving speed in the return processing gradually increases and reaches the maximum value of the moving speed represented by tan (Δθ3)×insertion length, the moving speed is restricted by the maximum value even when the moving speed does not reach the upper limit value of 0.01 mm/cycle. If the maximum value of the moving speed is greater than the upper limit, the moving speed is restricted by the upper limit. The upper limit is set within a range that does not cause the operator to feel that something is wrong.

In this embodiment, the first controller 310 performs the return processing at a moving speed smaller than the moving speed of the robot arm 50 moved through operation of the joystick 62 irrespective of whether the direction of movement of the robot arm 50 moved through operation of the joystick 62 is equal to or different from the direction of movement of the robot arm 50 in the return processing. For example, when the robot arm 50 is moved in a predetermined direction through operation of the joystick 62, the provisional pivot position PP3 is returned at a relatively small moving speed to the pivot position PP stored in the storage 351 irrespective of whether the direction of movement of the robot arm 50 moved in the return processing coincides with the predetermined direction or differs from the predetermined direction. Even when the direction of movement of the robot arm 50 moved through operation of the joystick 62 changes between the predetermined direction and a direction different from the predetermined direction, the deviated surgical instrument 1 is returned at a relatively small moving speed in return of the surgical instrument for making it pass through the pivot position PP.

Accordingly, in this embodiment, the first controller 310 performs the return processing at a moving speed smaller than the moving speed of the robot arm 50 moved through operation of the joystick 62 when the robot arm 50 restarts after temporarily stopping if the surgical instrument 1 is deviated from the pivot position PP stored in the storage 351.

Subsequently, in step S34, it is determined whether the provisional pivot position PP3 coincides with the pivot position PP stored in the storage 351. If it is determined that the provisional pivot position PP3 coincides with the pivot position PP in step S34, the return processing ends. Steps S31 to S34 are repeated until the provisional pivot position PP3 coincides with the pivot position PP. Operations from steps S31 to S34 are executed in each control cycle of the first controller 310.

### (Case of Insertion of Surgical Instrument)

In this embodiment, the first controller 310 does not perform the return processing when the robot arm 50 is moved to insert the surgical instrument 1 into the body of the patient P, and performs the return processing when the surgical instrument 1 is located inside the body of the patient P. Specifically, the surgical instrument 1 is inserted into the body of the patient P when the operator operates the linear switch 63 on the arm operation unit 60. When the robot arm 50 is moved through operation of the linear switch 63, the first controller 310 does not perform the return processing. The reason is to prevent the return processing from causing movement of the surgical instrument 1 in an oblique direction relative to a straight line when the operator attempts to move the surgical instrument 1 in the straight line, since the linear switch 63 is used to move the surgical instrument 1 in the straight line. If the surgical instrument 1 moves in such an oblique direction, the operator feels that something is wrong.

### (Case of Surgical Instrument Located Outside Patient's Body)

In this embodiment, the first controller 310 does not perform the return processing when the surgical instrument 1 is located outside the body of the patient P, and performs the return processing when the surgical instrument 1 is located inside the body of the patient P. The reason is that the patient P is not adversely affected even when the surgical instrument 1 is deviated from the pivot position PP during movement of the robot arm 50 in the case in which the surgical instrument 1 is located outside the body of the patient P. Examples of cases in which the surgical instrument 1 is located outside the body of the patient P include before a surgical operation and when the surgical instrument 1 is temporarily removed out of the body of the patient P for replacement of the surgical instrument 1.

### Advantages of the Embodiment

If the surgical instrument 1 is deviated from the pivot position PP stored in the storage 351, the first controller 310 performs the return processing to return the position of the robot arm 50 at a moving speed smaller than the moving speed of the robot arm 50 moved through operation of the joystick 62 so that the surgical instrument 1 passes through the pivot position PP. Accordingly, the robot arm 50 is returned to the position, which makes the surgical instrument pass through the pivot position, at a moving speed smaller than the moving speed of the robot arm 50 moved through operation of the joystick 62 in the return processing. As a result, because the moving speed of the robot arm 50 in a direction different from the operator's intended direction is reduced, the operator is unlikely to perceive movement of the robot arm 50 in the operator's unintended direction. Consequently, it is possible to prevent confusion of the operator caused by movement of the robot arm 50 in a direction different from the operator's intended direction 50 when the operator moves the robot arm through operation of the joystick 62.

The first controller 310 sets the maximum value of the moving speed in the return processing based on the moving speed of the robot arm 50 moved through operation of the joystick 62 and the insertion length of the surgical instrument 1 inserted into the patient P. Even when the provisional pivot position PP3 is moved by the same distance, the rotational moving amount of the surgical instrument 1 varies depending on the insertion length. To address this, since the maximum value of the moving speed is set based on the moving speed of the robot arm 50 and the insertion length of the surgical instrument 1 inserted into the patient P in return of the surgical instrument 1 for making it pass through the pivot position PP, it is possible to appropriately set the maximum value in accordance with the insertion length.

The upper limit of the maximum value of the moving speed in the return processing is previously set. Accordingly, even when the moving speed in the return processing increases, the operator can be prevented from feeling that something is wrong by setting the upper limit within a range that does not cause the operator to feel that something is wrong.

The first controller 310 performs the return processing at a moving speed smaller than the moving speed of the robot arm 50 moved through operation of the joystick 62 irrespective of whether the direction of movement of the robot arm 50 moved through operation of the joystick 62 coincides with or differs from the direction of the robot arm 50 in the return processing. Accordingly, it is possible to perform the return processing without confusion of the operator irrespective of whether the direction of movement of the robot arm 50 moved through operation of the joystick 62 coincides with or differs from the direction of the robot arm 50 in the return processing.

The robotic surgical system 500 includes the display 22a of the input 22 that receives a setting of the degree of moving speed of the robot arm 50. The first controller 310 performs the return processing at a moving speed smaller than the moving speed of the robot arm 50 based on the degree of moving speed set through the display 22a if the surgical instrument 1 is deviated from the pivot position PP stored in the storage 351. Accordingly, it is possible to perform the return processing without confusion of the operator even when the degree of moving speed of the robot arm 50 is changed through the display 22a.

The first controller 310 performs the return processing at a moving speed smaller than the moving speed of the robot arm 50 moved through operation of the joystick 62 when the robot arm 50 restarts after temporarily stopping if the surgical instrument 1 is deviated from the pivot position PP stored in the storage 351. Accordingly, it is possible to prevent the robotic surgical system 500 from being used with the surgical instruments 1 deviated from the pivot position PP after the robotic surgical system 500 restarts.

The first controller 310 does not perform the return processing when the robot arm 50 is moved to insert the surgical instrument 1 into the body of the patient P, and performs the return processing when the surgical instrument 1 is located inside the body of the patient P. If the return processing is performed when the surgical instrument 1 is inserted into the body of the patient P, the surgical instrument 1 may be moved in a direction different from the direction in which the surgical instrument 1 is inserted into the body of the patient P. To address this, the first controller does not perform the return processing when the robot arm 50 is moved to insert the surgical instrument 1 into the body of the patient P to prevent the surgical instrument 1 from moving in a direction different from the operator's intended direction in insertion of the surgical instrument into the body of the patient P.

The first controller 310 does not perform the return processing when the surgical instrument 1 is located outside the body of the patient P, and performs the return processing when the surgical instrument 1 is located inside the body of the patient P. The patient P is not adversely affected even when the surgical instrument 1 is deviated from the pivot position PP during movement of the robot arm 50 in the case in which the surgical instrument 1 is located outside the body of the patient P. For this reason, the load of controlling the first controller 310 can be reduced by not performing the return processing when the surgical instrument 1 is located outside the body of the patient P.

The first controller 310 gradually increases the moving speed in the return processing. Accordingly, it is possible to speedily perform the return processing as compared to the case in which the moving speed during the return processing is kept constant at the initial slow speed.

### Modified Embodiments

Note that the embodiment disclosed this time must be considered as illustrative in all points and not restrictive. The scope of the present invention is not shown by the above description of the embodiments but by the scope of claims for patent, and all modifications or modified examples within the meaning and scope equivalent to the scope of claims for patent are further included.

While the example in which the pivot button 66 is arranged on the arm operation unit 60 attached to the robot arm 50 has been shown in the aforementioned embodiment, the present disclosure is not limited to this. For example, the pivot button 66 may be directly arranged on the robot arm 50.

While the example in which the joystick 62 arranged on the arm operation unit 60 is used as an operation tool for controlling the movement of the surgical instrument 1 moved by the robot arm 50 has been shown in the aforementioned embodiment, the present disclosure is not limited to this. For example, another operation tool other than the joystick 62 may be used as the operation tool.

While the example in which the maximum value of the moving speed in the return processing is set based on the moving speed of the robot arm 50 moved through operation of the joystick 62 and the insertion length of the surgical instrument 1 inserted into the patient P has been shown in the aforementioned embodiment, the present disclosure is not limited to this. For example, the maximum value of the moving speed may be set based on only the moving speed of the robot arm 50 or the insertion length of the surgical instrument 1 inserted into the patient P.

While the example in which the upper limit of the maximum value of the moving speed in the return processing is previously set has been shown in the aforementioned embodiment, the present disclosure is not limited to this. For example, the upper limit may not be set as long as the maximum value of the moving speed in the return processing does not excessively increase.

While the example in which the first controller 310 performs the return processing irrespective of whether the direction of movement of the robot arm 50 moved through operation of the joystick 62 coincides with or differs from the direction of movement of the robot arm 50 in the return processing has been shown in the aforementioned embodiment, the present disclosure is not limited to this. For example, the first controller 310 may perform the return processing only when the difference between the direction of movement of the robot arm 50 moved by the operation of the joystick 62 and the direction of movement of the robot arm 50 in the return processing is greater than a predetermined threshold.

While the example in which the degree of moving speed of the robot arm 50 can be changed has been shown in the aforementioned embodiment, the present disclosure is not limited to this. For example, the degree of moving speed of the robot arm 50 may be fixed and unchangeable.

While the example in which the return processing is performed when the robot arm 50 restarts after temporarily stopping due to interference with the robot arm 50 or the like has been shown in the aforementioned embodiment, the present disclosure is not limited to this. For example, when the surgical instrument 1 is deviated from the pivot position PP while the robot arm 50 is in operation, the return processing may be performed while the robot arm 50 does not stop and is kept in operation.

While the example in which the first controller 310 does not perform the return processing when the robot arm 50 is moved to insert the surgical instrument 1 into the body of the patient P has been shown in the aforementioned embodiment, the present disclosure is not limited to this. For example, the first controller 310 may also perform the return processing when the robot arm 50 moves to insert the surgical instrument 1 into the body of the patient P.

While the example in which the first controller 310 does not perform the return processing when the surgical instrument 1 is located outside the body of the patient P has been shown in the aforementioned embodiment, the present disclosure is not limited to this. For example, the first controller 310 may perform the return processing even when the surgical instrument 1 is located outside the body of the patient P. That is, the first controller 310 may automatically move the surgical instrument 1 located outside the body of the patient P to return the pivot position PP to its original position.

While the example in which the first controller 310 gradually increases the moving speed in the return processing has been shown in the aforementioned embodiment, the present disclosure is not limited to this. For example, the moving speed in the return processing may be fixed.

While the example in which the program 311a is stored in the storage 311 has been shown in the aforementioned embodiment, the present disclosure is not limited to this. For example, the program 311a may be previously stored in an external memory or other storage medium, and the program 311a stored in the external memory may be then stored into the storage 311.

While the example in which the first controller 310 provided in the medical cart 10 is used as a controller of the present disclosure has been shown in the aforementioned embodiment, the present disclosure is not limited to this. In the present disclosure, a control device other than the first controller 310 may be used as the controller of the present disclosure.

While the example in which the four robot arms 50 are provided has been shown in the aforementioned embodiment, the present disclosure is not limited to this. In the present disclosure, the robot arms 50 may be provided in any number other than four.

While the example in which the arm parts 51 and the positioner 30 are constructed of a 7-axis multi-joint robot has been shown in the aforementioned embodiment, the present disclosure is not limited to this. For example, the arm parts 51 and the positioner 30 are constructed of a multi-joint robot having an axis configuration other than the 7-axis multi-joint robot. The multi-joint robot having an axis configuration other than the 7-axis multi-joint robot can be a 6-axis or 8-axis multi-joint robot, for example.

While the example in which the surgical robot 100 includes the medical cart 10, the positioner 30, the arm base 40 and the robot arms 50 has been shown in the aforementioned embodiment, the present disclosure is not limited to this. The medical cart 10, the positioner 30 and the arm base 40 are not necessarily provided, and the surgical robot 100 may include only the robot arms 50, for example.

Functions of elements disclosed in this specification can be realized by a circuit or processing circuit including a general purpose processor, a dedicated processor, an integrated circuit, ASIC (Application Specific Integrated Circuits), a conventional circuit and/or combination of them configured or programmed to realize the functions disclosed. A processor is considered as a processing circuit or circuits because it contains transistors and other circuitry. In the present disclosure, a circuit, unit, or means is hardware that performs an enumerated function or is hardware programmed to perform an enumerated function. The hardware may be the hardware disclosed herein or any other known hardware that is programmed or configured to perform the enumerated functions. If the hardware is a processor, which is considered as a type of circuit, the circuit, means, or unit is a combination of hardware and software, and software is used to configure the hardware and/or processor.

### [Modes]

The aforementioned exemplary embodiment will be understood as concrete examples of the following modes by those skilled in the art.

### [Mode 1]

A robotic surgical system includes a robot arm having a distal end to which a surgical instrument is attached; a pivot-position setter that stores, in a storage, a pivot position on the surgical instrument attached to the robot arm that serves as a pivot point during movement; an operation tool that controls movement of the surgical instrument moved by the robot arm; and a controller that performs return processing, if the surgical instrument is deviated from the pivot position stored in the storage, to return the robot arm to a position that makes the surgical instrument pass through the pivot position at a moving speed smaller than a moving speed of the robot arm moved through operation of the operation tool.

### [Mode 2]

In the robotic surgical system according to mode 1, the controller sets a maximum value of the moving speed in the return processing based on the moving speed of the robot arm moved through operation of the operation tool and an insertion length of the surgical instrument inserted into a patient.

### [Mode 3]

In the robotic surgical system according to mode 2, an upper limit of the maximum value is previously set.

### [Mode 4]

In the robotic surgical system according to any of modes 1 to 3, the controller performs the return processing at a moving speed smaller than the moving speed of the robot arm moved through operation of the operation tool irrespective of whether a direction of movement of the robot arm moved through operation of the operation tool coincides with or differs from a direction of movement of the robot arm in the return processing.

### [Mode 5]

In the robotic surgical system according to any of mode 1 to 4, a setting receiver that receives a setting of a degree of moving speed of the robot arm is further provided; and the controller performs the return processing at a moving speed smaller than the moving speed of the robot arm based on the degree of a moving speed set by the setting receiver if the surgical instrument is deviated from the pivot position stored in the storage.

### [Mode 6]

In the robotic surgical system any of mode 1 to 5, the controller performs the return processing at a moving speed smaller than the moving speed of the robot arm moved through operation of the operation tool when the robot arm restarts after temporarily stopping if the surgical instrument is deviated from the pivot position stored in the storage.

### [Mode 7]

In the robotic surgical system any of mode 1 to 6, the controller does not perform the return processing when the robot arm is moved to insert the surgical instrument into patient's body, and performs the return processing when the surgical instrument is located inside the patient's body.

### [Mode 8]

In the robotic surgical system any of mode 1 to 7, the controller does not perform the return processing when the surgical instrument is located outside the patient's body and performs the return processing when the surgical instrument is located inside the patient's body.

### [Mode 9]

In the robotic surgical system any of mode 1 to 7, the controller gradually increases the moving speed in the return processing.

### [Mode 10]

A surgical robot includes a robot arm having a distal end to which a surgical instrument is attached; a pivot-position setter that stores, in a storage, a pivot position on the surgical instrument attached to the robot arm that serves as a pivot point during movement; an operation tool that controls movement of the surgical instrument moved by the robot arm; and a controller that performs return processing, if the surgical instrument is deviated from the pivot position stored in the storage, to return the robot arm to a position that makes the surgical instrument pass through the pivot position at a moving speed smaller than a moving speed of the robot arm moved through operation of the operation tool.

### [Mode 11]

A method for controlling a robotic surgical system includes storing, in a storage, a pivot position on a surgical instrument attached to a distal end of a robot arm that serves as a pivot point during movement; and returning the robot arm to a position that makes the surgical instrument pass through the pivot position at a moving speed smaller than a moving speed of the robot arm moved through operation of an operation tool that is operated to move the surgical instrument by using the robot arm if the surgical instrument is deviated from the pivot position stored in the storage.

### [Mode 12]

A storage medium stores a program, the program including instructions for storing, in a storage, a pivot position on a surgical instrument attached to a distal end of a robot arm that serves as a pivot point during movement; and for returning the robot arm to a position that makes the surgical instrument pass through the pivot position at a moving speed smaller than a moving speed of the robot arm moved through operation of an operation tool that is operated to move the surgical instrument by using the robot arm if the surgical instrument is deviated from the pivot position stored in the storage.

What is claimed is:

1. A robotic surgical system comprising:
a robot arm having a distal end to which a surgical instrument is attached;
a pivot-position setter that stores, in a storage, a pivot position on the surgical instrument attached to the robot arm that serves as a pivot point during movement;
an operation tool that controls movement of the surgical instrument moved by the robot arm; and
a controller that performs return processing, if the surgical instrument is deviated from the pivot position stored in the storage, to return the robot arm to a position that makes the surgical instrument pass through the pivot position at a moving speed smaller than a moving speed of the robot arm moved through operation of the operation tool.

2. The robotic surgical system according to claim 1, wherein the controller sets a maximum value of the moving speed in the return processing based on the moving speed of the robot arm moved through operation of the operation tool and an insertion length of the surgical instrument inserted into a patient.

3. The robotic surgical system according to claim 2, wherein an upper limit of the maximum value is previously set.

4. The robotic surgical system according to claim 1, wherein the controller performs the return processing at a moving speed smaller than the moving speed of the robot arm moved through operation of the operation tool irrespective of whether a direction of movement of the robot arm moved through operation of the operation tool coincides with or differs from a direction of movement of the robot arm in the return processing.

5. The robotic surgical system according to claim 1 further comprising a setting receiver that receives a setting of a degree of moving speed of the robot arm, wherein the controller performs the return processing at a moving speed smaller than the moving speed of the robot arm based on the degree of a moving speed set by the setting receiver if the surgical instrument is deviated from the pivot position stored in the storage.

6. The robotic surgical system according to claim 1, wherein the controller performs the return processing at a moving speed smaller than the moving speed of the robot arm moved through operation of the operation tool when the robot arm restarts after temporarily stopping if the surgical instrument is deviated from the pivot position stored in the storage.

7. The robotic surgical system according to claim 1, wherein the controller does not perform the return processing when the robot arm is moved to insert the surgical instrument into patient's body, and performs the return processing when the surgical instrument is located inside the patient's body.

8. The robotic surgical system according to claim 1, wherein the controller does not perform the return processing when the surgical instrument is located outside patient's body and performs the return processing when the surgical instrument is located inside the patient's body.

9. The robotic surgical system according to claim 1, wherein the controller gradually increases the moving speed in the return processing.

10. A surgical robot comprising:
a robot arm having a distal end to which a surgical instrument is attached;
a pivot-position setter that stores, in a storage, a pivot position on the surgical instrument attached to the robot arm that serves as a pivot point during movement;
an operation tool that controls movement of the surgical instrument moved by the robot arm; and
a controller that performs return processing, if the surgical instrument is deviated from the pivot position stored in the storage, to return the robot arm to a position that makes the surgical instrument pass through the pivot position at a moving speed smaller than a moving speed of the robot arm moved through operation of the operation tool.

11. A method for controlling a robotic surgical system comprising:
storing, in a storage, a pivot position on a surgical instrument attached to a distal end of a robot arm that serves as a pivot point during movement; and
returning the robot arm to a position that makes the surgical instrument pass through the pivot position at a moving speed smaller than a moving speed of the robot arm moved through operation of an operation tool that is operated to move the surgical instrument by using the robot arm if the surgical instrument is deviated from the pivot position stored in the storage.

12. A storage medium storing a program, the program comprising instructions
for storing, in a storage, a pivot position on a surgical instrument attached to a distal end of a robot arm that serves as a pivot point during movement; and
for returning the robot arm to a position that makes the surgical instrument pass through the pivot position at a moving speed smaller than a moving speed of the robot arm moved through operation of an operation tool that is operated to move the surgical instrument by using the robot arm if the surgical instrument is deviated from the pivot position stored in the storage.
